(19) United States
(12) Patent Application Publication
(10) Pub. No.: US 2001/0011085 A1
(43) Pub. Date: Aug. 2, 2001

(54) 4-SUBSTITUTED CEPHEM DERIVATIVES AS ELASTASE INHIBITORS

(76) Inventors: MARCO ALPEGIANI, MILAN (IT); PIERLUIGI BISSOLINO, SAN GIORGIO LOMELLINA (IT); MASSIMILIANO PALLADINO, MILAN (IT); ETTORE PERRONE, MILAN (IT)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(21) Appl. No.: 08/894,089

(22) PCT Filed: Nov. 23, 1996
(86) PCT No.: PCT/EP96/05227

§ 371 Date: None

(30) Foreign Application Priority Data
Dec. 15, 1995 (GB) 9525697.0

(51) Int. Cl. A61K 31/545; C07D 501/00; C07D 501/14

(52) U.S. Cl. 514/201; 514/202; 514/203; 514/204; 514/205; 514/206; 514/207; 514/209; 540/215; 540/221; 540/223; 540/224; 540/225; 540/226; 540/227; 540/228; 540/230

(57) ABSTRACT

The present invention relates to cephem-derivatives and to a process for their preparation, having proper substituents at C-2 position, i.e. heterocyclylthio or acyloxy group. They are potent protease inhibitors, in particular human leucocyte elastase (HLE) inhibitors.

These inhibitors can be synthesised by way of a substitution reaction starting from known cephem compounds having a 2-position a suitable leaving group.

The present invention relates to new cephem derivatives, their preparation, and to pharmaceutical and veterinary compositions containing them. A peculiar structural feature of the cephem derivatives herein described is the simultaneous presence of a heterocyclyl-thio group, or an acyloxy group, at the C-2 position of the cephem ring (—X in formula I herebelow), and of an ester, thioester or amide group at the C-4 position (—COQ in formula I).

According to the invention there are provided cephem sulfoxides or sulfones of formula (I) and the pharmaceutically and veterinarily acceptable salts thereof

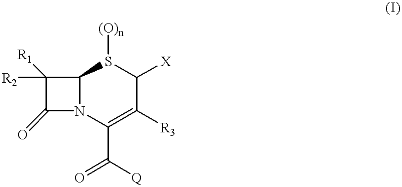

wherein n is one or two;

X is

(1) a heterocyclyl-thio group, wherein the heterocyclyl group is an optionally substituted 3-6 membered, saturated or unsaturated heterocyclic ring, containing at least one heteroatom selected from O, S and N, which is optionally fused to a second 5-6 membered, saturated or unsaturated heterocyclyl group, or to a C₃-C₈ cycloalkyl group, or to a cyclopentenyl group, or to a C₆-C₁₀ aryl group;

(2) an acyloxy group —O—C(O)A wherein A is an organic radical selected from C₁-C₁₂ straight or branched alkyl, C₂-C₁₂ alkenyl, C₂-C₁₂ alkynyl, C₆-C₁₄ aryl, C₃-C₈ cycloalkyl, C₅-C₈ cycloalkenyl, or C₇-C₁₈ aralkyl, C₈-C₁₈ aralkenyl, C₈-C₁₈ aralkynyl, (cycloalkyl)alkyl, (cycloalkyl)alkenyl, heterocyclyl, (heterocyclyl)alkyl, (heterocyclyl)alkenyl; Q represents a group —OA, —SA or —NAA′ wherein A is as defined above and A′ is hydrogen or, being the same or different, is as defined above for A; or A and A′ taken together with the nitrogen atom to which they are attached represent a 5-7 membered ring optionally containing an additional heteroatom selected from N, O and S;

R¹ and R² independently represent

(1) hydrogen, chloro, fluoro, bromo or iodo;

(2) A as defined above;

(3) hydroxy —OH or an ether —OA wherein A is as defined above;

(4) a thioether, sulfoxide or sulfone —S(O)ₘA wherein m is either zero, one or two and A is as defined above;

(5) acyloxy —OC(O)A wherein A is as defined above;

(6) acyl —C(O)A or —C(O)OA wherein A is as defined above;

(7) sulfonyloxy —OS(O)₂A wherein A is as defined above;

(8) acetylamino or trifluoroacetamido, or an acylamino group ZNH—CHR—CONH—, wherein R is methyl, ethyl, isopropyl Me₂CH—, EtMeCH—, Me₂CHCH₂—, EtMeCHCH₂, and Z is either hydrogen or:

phenylcarbonyl or phenoxymethylcarbonyl or phenoxy-methylcarbonyl, wherein the phenyl ring is either unsubstituted or substituted by one or two chloro, fluoro or methyl groups, or by a group selected among the following ones:

a) carboxy

b) OCH₂CO₂H

c) OCH₂CH₂-4-morpholinyl

d) OCH₂CH₂-1-pyrrolidinyl

e) SO₂-1-morpholinyl

f) SO₂-(4-methyl)-1-piperazinyl

g) SON(Me)CH₂CH₂NMe₂

h) CONH—CO-1-morpholinyl

i) CO-1-morpholinyl

j) CO-(4-methyl)-1-piperazinyl

k) CONH—CH₂CH₂-1-morpholinyl

l) COO—CH₂CH₂-1-morpholinyl

m) SO₂NH—Ar, or CONHSO₂—Ar wherein Ar is a

phenyl ring either unsubstituted or substituted by Cl, F or carboxy, said carboxy being optionally as the ethyl ester COOC₂H₅ or amides CONH₂, CONHCH₃, CONHCH₂ CO₂H;

an amino-blocking group, selected from the group consisting of e), f), g), i), k), l) above or from the following ones: methyl, dimethyl, benzyl, CO₂CH₃, COSCH₃; SO₂CH₃;

(9) azido, nitro or cyano;

or R¹ and R² taken together constitute an oxo group (═O) or a group of formula ═CHA′, ═CHC(O)A′, ═CHC(O)OA′ or ═CHS(O)₂A wherein A and A′ are as defined above;

R³ represents:

(1) A′ as defined above;

(2) chloro or fluoro;

(3) a sulfenyl, sulfinyl or sulfonyl group —S(O)ₘA wherein m and A are as defined above;

(4) an ether group —O—A wherein A is as defined above;

(5) an acyl group —C(O)A, —C(O)OA or —CO₂H wherein A is as defined above;

(6) an oxymethyl group —CH₂—OA′ wherein A′ is as defined above;

(7) a thiomethyl group or a derivative thereof of formula —CH₂S(O)ₘA wherein m and A are as defined above;

(8) an acyloxymethyl group —CH₂OC(O)A′ wherein A′ is as defined above;

(9) an acylthiomethyl group —CH₂SC(O)A wherein A is as defined above;

(10) carbamoyloxymethyl —CH₂OCONH₂ or carbamoylthiomethyl (i.e., —CH₂SCONH₂), and the N-methyl and N,N-dimethyl derivatives thereof;

(11) an aminomethyl group —CH₂—N(A)A′ wherein A and A′ are as defined above;

(12) ammoniomethyl —CH₂N⁺(A)(A′)A″ wherein A and A′ are as defined above and A″, being the same or different, is as defined for A; or A is alkyl and A′ and A″ together with the nitrogen atom to which they are attached represent a 5-7 membered ring containing one nitrogen and optionally one oxygen or sulfur atom;

(13) an acylaminomethyl group —CH₂NH—C(O)A wherein A is as defined above.

A 3-6 membered, saturated or insaturated heterocyclyl ring is, for example, pyrrolyl, pyrrolidyl, pyrrolinyl, pyrazolyl, pyrazolidyl, pyrazolinyl, imidazolyl, imidazolidyl, imidazolinyl, triazolyl, tetrazolyl, oxazolyl, oxazolidyl, oxazolinyl, isoxazolyl, isoxazolidyl, isoxazolinyl, thiazolyl, thiazolidyl, thiazolinyl, isothiazolyl, isothiazolidyl, isothiazolinyl, thiadiazolyl, thienyl, furyl, aziridinyl, oxiranyl, aziridinyl, pyridinyl, piperidyl, pyrazinyl, pyrimidinyl, triazinyl, pyranyl, pyridazinyl, morpholinyl, thiamorpholinyl. A fused heterocyclyl ring is, for example, benzothienyl, benzothiazolyl, benzoxazolyl, isobenzofuranyl, benzofuranyl, chromenyl, indolyl, indolizinyl, isoindolyl, cinnolinyl, indazolyl, purinyl, benzopyridyl, benzopiperidyl, pyrindinyl, dihydro-pyrindinyl, (tetrazolo)pyridazinyl.

A C₁-C₁₂ alkyl group is a straight or branched alkyl group such as methyl, ethyl, n-propyl, isopropyl, n-butyl, isobutyl, sec-butyl, tert-butyl, n-pentyl, n-hexyl and so on.

A C₂-C₁₂ alkenyl group is a straight or branched alkenyl group such as vinyl, allyl, crotyl, 2-methyl-1-propenyl, 1-methyl-1-propenyl, butenyl, pentenyl and so on. A C₂-C₁₂ alkynyl group is a straight or branched alkynyl group such as ethynyl, propargyl, 1-propynyl, 1-butynyl, 2-butynyl and so on.

A C₆-C₁₄ aryl group is a monocyclic, bicyclic or tricyclic aromatic hydrocarbon group of 6 to 14 carbon atoms, such as phenyl, naphthyl, phenanthryl or anthryl.

A C₃-C₈ cycloalkyl group is a saturated carbocyclic group of 3 to 6 carbon atoms, such as cyclopropyl, cyclobutyl, cyclopentyl, cyclohexyl and so on.

A C₅-C₈ cycloalkenyl group is an unsaturated carbocyclic group such as cyclopentenyl, cyclohexenyl and so on. A C₇-C₁₈ aralkyl group is an alkyl group of 1 to 4 carbon atoms linked to a monocyclic, bicyclic or tricyclic aromatic hydrocarbon group of 6 to 14 carbon atoms. Examples of aralkyl groups are benzyl, phenylethyl, naphthylmethyl and anthrylmethyl.

A C₈-C₁₈ aralkenyl group is an alkenyl group of 2 to 4 carbon atoms linked to a monocyclic, bicyclic or tricyclic aromatic hydrocarbon group of 6 to 14 carbon atoms. Examples of aralkenyl groups are styryl, 2-phenyl-1-propenyl, 3-phenyl-2-butenyl, 2-naphthylethenyl, anthrylethenyl and so on.

A C₈-C₁₈ aralkynyl group is an alkynyl group of 2 to 4 carbon atoms linked to a monocyclic, bicyclic or tricyclic aromatic hydrocarbon group of 6 to 10 carbon atoms. Examples of aralkynyl groups are 2-phenylethynyl, 2-naphthylethynyl, anthrylethynyl and so on.

A (cycloalkyl)alkyl group is an alkyl group of 1 to 4 carbon atoms linked to a C₃-C₈ cycloalkyl group defined above. A (cycloalkyl)alkenyl group is an alkenyl group of 2 to 4 carbon atoms linked to a C₃-C₈ cycloalkyl group defined above.

A heterocyclyl group is a 3-6 membered, saturated or unsaturated heterocyclyl ring as defined above, which is optionally fused to a second 5-6 membered heterocyclyl ring, a C₃-C₈ cycloalkyl group, a cyclopentenyl group, or to a C₆-C₁₀ aryl group defined above.

A (heterocyclyl)alkyl group is an alkyl group of 1 to 4 carbon atoms linked to a heterocyclyl group defined above. A (heterocyclyl)alkenyl group is an alkenyl group of 2 to 4 carbon atoms linked to a heterocyclic group defined above. The term halogen (or halo) preferably encompasses fluorine, chlorine or bromine.

The above said alkyl, alkenyl, alkynyl, cycloalkyl, cycloalkenyl, aryl, aralkyl, aralkenyl, aralkynyl, (cycloalkyl)alkyl, (cycloalkyl)alkenyl, heterocyclyl, (heterocyclyl)alkyl, (heterocyclyl)alkenyl groups can be either unsubstituted or substituted by one or more substituents selected from the following ones:

halo (i.e., fluoro, bromo, chloro or iodo);

hydroxy;

nitro;

azido;

mercapto (—SH);

amino (i.e., —NH₂, or —NHRⁱ or —NRⁱRⁱⁱ wherein Rⁱ and Rⁱⁱ, which are the same or different, are C₁-C₁₂ straight or branched alkyl or phenyl or benzyl);

formyl (i.e., —CHO);

cyano;

oxo (i.e., ═O) or imino (i.e., ═NH);

carboxy(alkyl) (i.e., (CH₂)ₜCOOH or (CH₂)ₜCOORⁱ wherein Rⁱ is as defined above and t is 0, 1, 2 or 3);

sulfo (i.e., —SO₃H);

acyl (i.e., —C(O)Rⁱ wherein Rⁱ is as defined above) or tri-fluoroacetyl (i.e., —C(O)CF₃);

carbamoyl (i.e., —CONH₂); N-methylcarbamoyl (i.e., —CONHCH₃), N,N-dimethylcarbamoyl, or N-carboxymethyl-carbamoyl (i.e., —CONHCH₂COOH);

carbamoyloxy (i.e., —OCONH₂);

acyloxy (i.e., —OC(O)Rⁱ wherein Rⁱ is as defined above) or formyloxy (i.e., —OC(O)H);

alkoxycarbonyl or benzyloxycarbonyl (i.e., —C(O)ORⁱ wherein Rⁱ is as defined above);

alkoxycarbonyloxy or benzyloxycarbonyloxy (i.e., —OC(O)ORⁱ wherein Rⁱ is as defined above);

alkoxy, phenoxy or benzyloxy (i.e., —ORⁱ wherein Rⁱ is as defined above);

alkylthio, phenylthio or benzylthio (i.e., —SRⁱ wherein Rⁱ is as defined above);

alkylsulfinyl, phenylsulfinyl or benzylsulfinyl (i.e., —S(O)Rⁱ wherein Rⁱ is as defined above);

alkylsulfonyl, phenylsulfonyl or benzylsulfonyl (i.e., —S(O)₂Rⁱ wherein Rⁱ is as defined above);

acylamino —NHC(O)Rⁱⁱⁱ or —NHC(O)ORⁱⁱⁱ wherein Rⁱⁱⁱ is C₁-C₁₂ straight or branched alkyl, phenyl, benzyl, CH₂CH₂COOH or CH₂CH₂CH₂COOH;

sulfonamido (i.e., —NHSO₂Rⁱ wherein Rⁱ is as defined above);

sulfamoylamino (i.e., —NHSO₂NH₂);

ureido (i.e., —NHCONH₂);

guanidino (i.e., —NHC(═NH)NH₂);

C₁-C₄ alkyl, C₂-C₄ alkenyl or alkynyl;

C₃-C₆ cycloalkyl;

substituted methyl selected from chloromethyl, fluoromethyl, difluoromethyl, trifluoromethyl, aminomethyl, N,N-dimethylaminomethyl, azidomethyl, cyanomethyl, carboxy-methyl, sulfomethyl, carbamoylmethyl, carbamoyloxymethyl, hydroxymethyl, C₁-C₄ alkyloxycarbonylmethyl, guanidinomethyl.

The present invention also includes derivatives of compounds of formula 1 that have protecting groups at one or more carboxy, amino, hydroxy or mercapto groups. The carboxyl-protecting groups possibly present may be lower alkyl groups such as methyl, ethyl, propyl, isopropyl or tert-butyl; halogenated lower alkyl groups such as 2,2,2-trichoroethyl or 2,2,2-trifluoroethyl; lower alkanoyloxyalkyl groups such as acetoxymethyl, propionyloxymethyl, pivaloyloxymethyl, 1-acetoxyetyl, 1-propionyloxyethyl; lower alkoxycarbonyloxyalkyl groups such as 1-(methoxycarbonyloxy)ethyl, 1-(ethoxycarbonyloxy)ethyl, 1-(isopropoxycarbonyloxy)ethyl; lower alkenyl groups such as 2-propenyl, 2-chloro-2-propenyl, 3-methoxycarbonyl-2-propenyl, 2-methyl-2-propenyl, 2-butenyl, cinnamyl; aralkyl groups such as benzyl, p-methoxybenzyl, 3,4-dimethoxybenzyl, o-nitrobenzyl, p-nitrobenzyl, benzhydryl, bis(p-methoxyphenyl)methyl; (5-substituted 2-oxo-1,3-dioxol-4-yl)methyl groups such as (5-methyl-2-oxo-1,3-dioxol-4-yl)methyl; lower alkylsilyl groups such as trimethylsilyl, tert-butyldimethylsilyl, tert-butyldiphenylsilyl, triphenylsilyl; or an indanyl group, a phthalidyl group, a pyranyl group, a methoxymethyl or methylthiomethyl or a 2-methoxyethoxymethyl group. Particularly preferred are a tert-butyl group, a p-nitrobenzyl group, a p-methoxybenzyl group, a benzhydryl group, a tert-butyldimethylsilyl, tert-butyldiphenylsilyl group or a propenyl group.

The amino, hydroxy or mercapto protecting groups possibly present may be those usually employed in the chemistry of penicillins and cephalosporins for this kind of functions. They may be, for instance, optionally substituted, especially halo-substituted, acyl groups, e.g. acetyl, monochloroacetyl, dichloroacetyl, trifluoroacetyl, benzoyl or p-bromophenacyl; triarylmethyl groups, e.g. triphenylmethyl; silyl groups, in particular trimethylsilyl, dimethyl-tert-butylsilyl, diphenyl-tert-butylsilyl; or also groups such as tert-butoxycarbonyl, p-nitrobenzyloxycarbonyl, 2,2,2-trichloroethoxycarbonyl, benzyl and pyranyl. Preferred protecting groups of the hydroxy function are p-nitrobenzyloxycarbonyl; allyloxycarbonyl; dimethyl-tert-butylsilyl; diphenyl-tert-butylsilyl; trimethylsilyl; 2,2,2-trichloroethoxycarbonyl; benzyl; dimethoxybenzyl; p-methoxybenzyloxycarbonyl; p-bromophenacyl; triphenylmethyl, pyranyl, methoxymethyl, benzhydryl, 2-methoxyethoxymethyl, formyl, acetyl, trichloroacetyl. The present invention also includes salts of those compounds of formula (I) that have salt-forming groups, especially the salts of the compounds having a carboxylic group, a basic group (e.g. an amino or guanidino group), or a quaternary ammonium group. The salts are especially physiologically tolerable salts, for example alkali metal and alkaline earth metal salts (e.g. sodium, potassium, lithium, calcium and magnesium salts), ammonium salts and salts with an appropriate organic amine or amino acid (e.g. arginine, procaine salts), and the addition salts formed with suitable organic or inorganic acids, for example hydrochloric acid, sulfuric acid, carboxylic and sulfonic organic acids (e.g. acetic, trifluoroacetic, p-toluensulfonic acid). Some compounds of formula (I) which contain a carboxylate and an ammonium group may exist as zwitterions; such salts are also part of the present invention.

Furthermore, physiologically hydrolysable esters, hydrates and solvates of compounds of formula (I) are included within the scope of the present invention. The physiologically hydrolizable esters of the compounds (I) may include, for example, methoxycarbonylmethyl, 1-methoxycarbonyloxy-1-ethyl, indanyl, phthalidyl, methoxymethyl, pivaloyloxymethyl, glycyloxymethyl, phenylglycyloxymethyl or 5-methyl-2-oxo-1,3-dioxolan-4-yl esters, and other physiologically hydrolysable esters which have been widely for penicillin and cephalosporin antibiotics: more preferably, methoxycarbonyloxymethyl, 1-methoxycarbonyloxy-1-ethyl, methoxymethyl or pivaloyloxymethyl; and most preferably, methoxycarbonyloxymethyl or methoxymethyl. Typical solvates of the cephalosporin compounds of formula (I) may include solvates with water miscible solvents, e.g. methanol, ethanol, acetone or acetonitrile; more preferably, ethanol.

The present invention also includes within its scope pharmaceutical and veterinary compositions comprising one or more of the compounds (I), or a pharmaceutically or veterinarily acceptable salt thereof, as active ingredients, in association with a pharmaceutically or a veterinarily acceptable diluent or carrier. Excipients or other additives may be present, if necessary.

The present invention encompasses all the possible stereoisomers as well as their racemic or optically active mixtures.

Particularly preferred compounds are compounds of formula (I) wherein

n is two;

X is

(1′) an optionally substituted heterocyclyl-thio group, wherein the heterocyclyl group is an unsaturated heterocyclyl ring chosen among pyrrolyl, pyrazolyl, imidazolyl, triazolyl, tetrazolyl, oxazolyl, isoxazolyl, thiazolyl, isothiazolyl, thiadiazolyl, thienyl, furyl, pyridinyl, pyrazinyl, pyrimidinyl, triazinyl, pyridazinyl, benzothienyl, benzothiazolyl, benzoxazolyl, isobenzofuranyl, benzofuranyl, chromenyl, indolyl, indolizinyl, isoindolyl, cinnolinyl, indazolyl, purinyl, benzopyridyl, benzopiperidyl, (cyclopentano)pyridyl, (cyclopenteno)pyridyl, (tetrazolo)pyridazinyl;

(2′) a group —OC(O)A wherein A is chosen among an optionally substituted C₁-C₁₂ straight or branched alkyl, C₂-C₁₂ straight or branched alkenyl, C₂-C₁₂ alkynyl, C₃-C₆ cycloalkyl, C₆-C₁₄ aryl, benzyl, diphenylmethyl, stiryl, 2-phenyl-2-propyl; or an optionally substituted heterocyclyl, wherein the heterocyclyl group is either one of the unsaturated heterocyclyl groups specified under (1′) immediately above, or it is a 3-6 membered saturated ring containing 1-3 heteroatoms selected from N, O and S, preferably pyrrolinyl, aziridinyl, piperidyl, oxiranyl, tetrahydro-pyranyl, morpholinyl;

the substituents for the heterocyclyl groups and for the groups A defined above being selected from fluoro, chloro, bromo, nitro, cyano, sulfo, carboxy, tetrazolyl, C₁-C₄ alkoxycarbonyl, carbamoyl, sulfamoyl, carbamoyloxy, hydroxy, C₁-C₄ alkoxy, benzyloxy, benzhydryloxy, phenoxy, acetoxy, pivaloyloxy, benzoxy, methylthio, phenylthio, methansulfonyl, benzensulfonyl, sulfomethyl, carboxymethyl, carboxyethyl, carboxypropyl, carboxymethylthio, carboxyphenyl C₆H₅—COOH, carboxybenzyl CH₂—C₆H₅—COOH, acetyl, trifluoroacetyl, benzoyl, pivaloyl, amino, dimethylamino, diethylamino, dimethylaminoethyl, formamido, acetamido, trifluoroacetamido, pivalamido, oxo, C₁-C₅ straight or branched alkyl, vinyl and allyl;

Q represents a group —OA, —SA or —NAA′ wherein A is defined above and A′ is hydrogen or, being the same or different, is as defined above for A; or A and A′ taken together with the nitrogen atom to which they are attached represent a saturated 5-6 membered ring optionally containing an additional heteroatom selected from O, S, and N; said ring or said groups A and A′ being unsubstituted or substituted by one or more of the substituents detailed above;

R¹ and R², each independently, are

(1′) hydrogen, chloro, fluoro or bromo;

(2′) straight or branched C₁-C₅ alkyl or C₁-C₅ alkenyl; still more preferably methyl, ethyl, propyl, isopropyl, allyl;

(3′) C₁-C₅ alkyloxy or C₆-C₁₀ aryloxy; still more preferably methoxy or phenoxy;

(4′) C₁-C₅ alkylthio or C₆-C₁₀ arylthio; still more preferably methylthio or phenylthio;

(5′) C₁-C₅ alkylsulfinyl, C₂-C₅ alkenylsulfinyl, C₆-C₁₀ arylsulfinyl; still more preferably methylsulfinyl or phenylsulfinyl;

(6′) C₁-C₅ alkylsulfonyl or C₁-C₁₀ arylsulfonyl; still more preferably methylsulfonyl or phenylsulfonyl;

(7′) C₁-C₅ alkylcarbonyl or C₆-C₁₀ arylcarbonyl; still more preferably acetyl or benzoyl;

(8′) C₁-C₅ alkylcarbonyloxy or C₆-C₁₀ arylcarbonyloxy; still more preferably acetoxy or benzoyloxy;

(9′) C₁-C₅ alkylsulfonyloxy or C₆-C₁₀ arylsulfonyloxy; still more preferably methansulfonyloxy or benzensulfonyloxy;

(10′) acetamido or trifluoroacetamido;

(11′) nitro, azido, cyano, formyloxy;

(12′) an acylamino group ZNH—CHR—CONH derived from an L-aminoacid, wherein Z is

phenoxycarbonyl

4-chlorophenoxycarbonyl

benzyloxycarbonyl

4-chlorobenzyloxycarbonyl

4-[(1-morpholinyl)carbonyl]benzyloxycarbonyl

(1-morpholinyl)carbonyl

4-[(2-carboxyphenylaminosulfonyl] benzyloxy carbonyl;

4-[(4-chloro)phenylsulphonylamino]benzoyl;

still more preferably R is methyl group;

or R¹ and R² taken together constitute an oxo group, or a methylene group, or a group of formula ═CHY or ═CHC(O)Y or ═CHC(O)OY or ═CHS(O),Y, wherein Y is C₁-C₅ alkyl, C₁-C₅ alkenyl, C₁-C₁₀ aryl, C₇-C₁₀ aralky or heterocyclyl, wherein heterocyclyl is one of the heterocyclyl groups detailed above in the definition (1′) of X; the substituents for the groups defined under (2′)-(9′) being selected from fluoro, chloro, bromo, nitro, cyano, sulfo, carboxy, tetrazolyl, C₁-C₄ alkoxycarbonyl, carbamoyl, sulfamoyl, carbamoyloxy, methansulfonyl, hydroxy, C₁-C₄ alkoxy, benzyloxy, benzhydryloxy, phenoxy, acetoxy, pivaloyloxy, benzoxy, methylthio, phenylthio, methansulfonyl, benzensulfonyl, sulfomethyl, carboxymethyl, carboxyethyl, carboxypropyl, carboxymethylthio, carboxyphenyl C₆H₅—COOH, carboxybenzyl CH₂—C₆H₅—COOH, acetyl, trifluoroacetyl, benzoyl, pivaloyl, amino, dimethylamino, diethylamino, dimethylaminoethyl, formamido, acetamido, trifluoroacetamido, pivalamido, oxo, C₁-C₅ straight or branched alkyl, vinyl and allyl;

R³ is either hydrogen or

(1′) methyl, ethyl, propyl, phenyl or benzyl, optionally substituted by a group selected from chloro, bromo, fluoro, hydroxy, carbamoyloxy, carboxy;

(2′) chloro;

(3′) methylthio;

(4′) methoxy or benzyloxy;

(5′) formyl, acetyl, benzoyl, carboxy, methoxycarbonyl, ethoxycarbonyl, tert-butoxycarbonyl or benzyloxycarbonyl;

(6′) methoxymethyl, ethoxymethyl, isopropoxymethyl; or benzyloxymethyl, phenoxymethyl, 3-pyridyloxymethyl wherein the phenyl and pyridyl rings are either unsubstituted or substituted by one group or two equal or different groups chosen from hydroxy, carboxy, amino, halogen and C₁-C₄ alkoxycarbonyl;

(7′) methylthiomethyl, methylsulfinylmethyl or methylsulfonyl-methyl; or a heterocyclylthiomethyl group wherein the heterocyclyl ring is one of those listed above in the definition (1′) of X, said groups being either unsubstituted or substituted by one or two, equal or different groups chosen from the following ones: hydroxy, oxo, amino, imino, methylamino, dimethylamino, acetylamino, sulfo, carboxy, carbamoyl, methylcarbamoyl, dimethylcarbamoyl, carbamoyloxy, dimethylaminomethyl, carboxymethyl, carboxymethylthio, cyano, cyanomethyl, nitro, methoxy, phenoxy, benzyloxy, benzhydryloxy, methylthio, methylsulfonyl, acetoxy, benzoxy, halogen or C₁-C₄ alkyl or alkenyl;

(8′) acetoxymethyl, benzoyloxymethyl, phenylacetoxymethyl or C₃-C₆ alkanoyloxymethyl wherein the above groups are either unsubstituted or substituted by one or more groups selected from carboxy, carboxymethyl, hydroxy, C₁-C₃ alkoxy, carbamoyl;

(9′) carbamoyloxymethyl —CH₂OCONH₂;

(10′) trialkylammoniomethyl wherein the alkyl group is chosen from methyl, ethyl or propyl; N-methylpyrrolidiniomethyl; N-methylpiperidiniomethyl; N-methylmorpholiniomethyl; pyridiniomethyl which is either unsubstituted or substituted on the heterocyclic ring by fluoro, chloro, methoxy, hydroxy, carboxy or carbamoyl;

and the pharmaceutically and veterinarily acceptable salts thereof and all of the possible stereoisomers e.g.: epimers, diastereoisomers, geometrical isomers, tautomers. Specific examples of the preferred compounds of the present invention are those listed in Table I.

TABLE 1

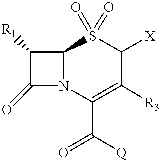

| No | R1 | R3 | X | Q |
|---|---|---|---|---|
| 1 | OCH₃ | CH₃ | 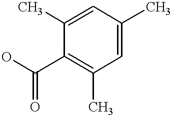 | 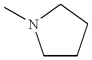 |
| 2 | ″ | ″ | ″ | 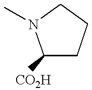 |
| 3 | ″ | ″ | 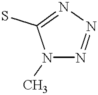 | ″ |
| 4 | ″ | ″ | ″ | 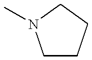 |
| 5 | ″ | ″ | ″ | —OBu^{t} |
| 6 | ″ | ″ | ″ | —SBu^{t} |
| 7 | ″ | ″ | —OCOPh | —OBu^{t} |
| 8 | ″ | ″ | ″ | —SBu^{t} |
| 9 | ″ | ″ | ″ | 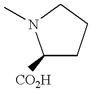 |
| 10 | ″ | ″ | ″ | 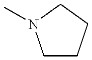 |
| 11 | OCH₃ | CH₃ | 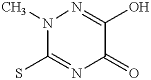 | 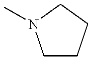 |
| 12 | ″ | ″ | ″ | 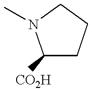 |
| 13 | ″ | ″ | ″ | —OBu^{t} |
| 14 | ″ | ″ | ″ | —SBu^{t} |
| 15 | ″ | ″ | —OCOBu^{t} | 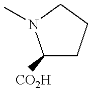 |
| 16 | ″ | ″ | —OCOCMe₂COOH | 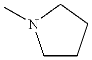 |
| 17 | CH₃CH₂ | ″ | 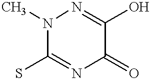 | 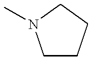 |
| 18 | ″ | ″ | 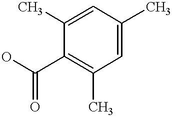 | 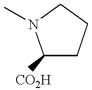 |
| 19 | OCH₃ | 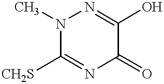 | 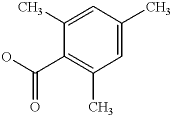 | 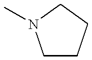 |
| 20 | OCH₃ | CH₃ | 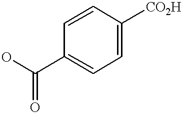 | 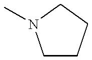 |
| 21 | ″ | ″ | ″ | 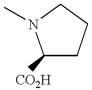 |
| 22 | ″ | ″ | 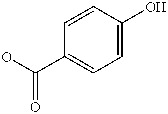 | ″ |
| 23 | ″ | ″ | 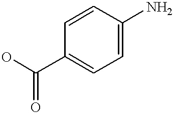 | 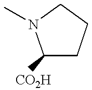 |
| 24 | ″ | ″ | 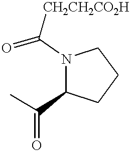 | 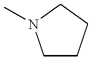 |
| 25 | ″ | 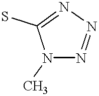 | 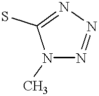 | 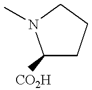 |

The present invention also provides a process for the preparation of cephem sulfones of formula (I), which process comprises:

(i) reacting a compound of formula (II)

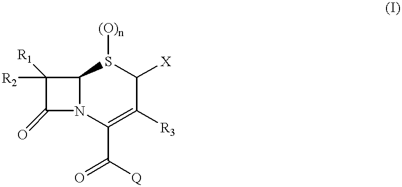

wherein either

(iₐ) n, Q, R¹, R² and R³ are as defined above, and L is a leaving group, with compounds of formula (III)

X-M (III)

wherein X is as defined above and N is hydrogen or a metal; or

(i_{b}) n, Q, R¹, R² and R³ are as defined above, and L is hydrogen, with compounds of formula (IV)

X-X′ (IV)

wherein X is as defined above and X′, being the same or different is as defined above for X, or a group selected from halogen, i.e. fluorine, chlorine, bromine or iodine, a C₁-C₈ alkylsulfonyl such as mesyl or triflyl, an arylsulfonyl such as tosyl or brosyl, an imido group such as succinimido or phthalimido, or a leaving group of formula —OC(Q)A, —OC(O)OA, —OS (O)₂A, —OC(O)NR^{iv}A wherein A is as defined above and R^{iv} is phenyl or a C₁-C₄ alkyl group;

(ii) if desired, oxidising a resulting compound of formula (I) wherein n is one into the corresponding compound wherein n is two;

(iii) if desired, converting the resulting compound of formula (I) into a pharmaceutical or veterinarily acceptable salt thereof.

In step (iₐ) the leaving group L of formula (II) is preferably a halogen atom, preferably bromine, chlorine or iodine. When M of formula (III) is hydrogen the reaction is usually performed in the presence of an inorganic or organic base. These external bases are generally not required when M of formula (III) is a metal, e.g an alkaline metal or a heavy metal, preferably a halophilic metal such as silver, copper, mercury, lead. The reaction can be carried out in a wide range of organic solvents such as acetonitrile, N,N-dimethylformamide, dichloromethane, tetrahydrofuran, dioxane, ethyl acetate, chloroform, benzene, carbon tetrachloride, ethyl ether, dimethoxyethane, sulfolane, dimethylsulfoxide, hexamethylphosphoramide, N-methyl pyrrolidone, acetone, water or mixtures thereof. Reaction temperatures range between −50° C. and +120° C., preferably between −20° C. and +80° C. Preferred external bases are tertiary organic bases either aliphatic or aromatic or alicyclic such as triethylamine, diisopropylethylamine, aniline, pyridine, lutidine, collidine, quinoline, N-methylmorpholine, N-methylpyrrolidine, 1,4-diazabicyclo[2,2,2]octane (DABCO); or inorganic bases such as alkaline bicarbonates, or carbonates, e.g. sodium bicarbonate, calcium carbonate, cesium carbonate, potassium carbonate. A beneficial effect may be observed upon addition of alkaline metal salts such as sodium iodide or potassium iodide and additives such as molecular sieves, alumina or calcium oxide. The reaction can also be carried out in the presence of heavy metal salts such as silver nitrate, silver perchlorate, silver triflate, copper nitrate, mercury nitrate.

Step (i_{b}) is usually performed in the presence of tertiary alyphatic or aromatic organic bases such as 1,5-diazabicyclo-[4,3,0]non-5-ene (DBN), 1,8-diazabicyclo[5,4,0]undec-7-ene (DBU), 1,1,3,3-tetramethylguanidine, 1,4-diazabicyclo[2,2,2]octane (DABCO), N,N-diisopropylethylamine, N-methylmorpholine, N-methylpyrrolidine, triethylamine, pyridine, lutidine, collidine, quinoline. The reaction can be carried out in a wide range of non-protic organic solvents such as acetonitrile, N,N-dimethylformamide, tetrahydrofuran, dioxane, benzene, sulfolane, N,N-dimethylacetamide, hexamethylphosphoramide, N-methylpyrrolidone or mixtures thereof. Reaction temperatures range between −60° C. and +40° C., preferably between −30° C. and room temperature. In step (ii), the oxidation of compounds of formula (I) wherein n=1 into the corresponding compounds wherein n=2 may be performed with organic or inorganic peracids or salts thereof, preferably peracetic acid, metachloroperbenzoic acid, permaleic acid, perphthalic acid, oxone, sodium or potassium persulfate. The reaction can be carried out in a wide range of organic solvents, or mixtures of organic solvents with water. Preferred reaction temperatures range between −40° C. and +40° C.

It is understood that in the process above any functional group, if needed or desired, can be masked by conventional methods at any stage and unmasked at the end or when convenient. It is also understood that the groups R¹, R², R³, X and Q can be converted by conventional methods into different groups included within those previously defined, if desired, at the end or at any stage of the process above. This conversion or masking/unmasking of the protecting groups can be performed by known methods, most of which are popular in the chemistry of cephalosporin antibiotics (see, e.g. “Cephalosporins and Penicillins”, E. H. Flynn Ed.). Compounds of formula (II) are known or can be prepared from known compounds. Compounds of formula (III) and (IV) are known compounds or can be prepared from known compounds by known methods.

The compounds of the present invention are characterized by high inhibitory activity on elastases, especially human leukocyte elastase (HLE). In particular, the distinctive substitution pattern born at C-2 by the compounds of formula (I) resulted in an unpredictable enhancement of inhibitory activity, relative to the corresponding C2-unsubstituted compounds, which were disclosed, for example, in EP-A-267723 and WO 89/10926. Thus, when tested as inhibitors of HLE, representative compounds of formula (I) showed good “potency” (low value of apparent dissociation constant of the HLE-inhibitor complex at steady state, Kᵢ^{ss}) and good “efficiency” (high value of rate of formation of the HLE-inhibitor complex, k₅/Kᵢ). These efficacy parameters for HLE inhibition, Kᵢ^{ss} and k₅/K_{i, are defined in the equation below, representing the interaction of the enzyme (E) with the compounds (I), which is monitored (see Protocol) in the presence of a chromogenic enzyme substrate (S):}

$${K}_{I} = \frac{{k}_{4}}{{k}_{3}}$$

$${K}_{I}^{SS} = {K}_{I} \frac{{k}_{6}}{( {k}_{5} + {k}_{6} )}$$

wherein

E=enzyme (HLE)

S=substrate (see Protocol)

P=product (see Protocol)

I=inhibitor (compounds of formula (I))

EI=initial enzyme:inhibitor complex

EI*=final enzyme:inhibitor adduct (inactivated enzyme)

I*=turnover product (inactivated inhibitor)

Protocol

Kinetic parameters of HLE (Elastin Product Company) were determined at 37° C., 0.027M pH 7.4 phosphate buffer, lot DMSO, 1% MeCN, NaCl (I=0.15), by monitoring the release of 7-amino-4-methylcoumarin (fluorescence detection) from N-methoxysuccinyl-alanyl-prolyl-valyl-7-amido-4-methylcoumarin as the substrate, according to the equations:

$$[ P ] = {V}_{s} t + \frac{{V}_{z} - {V}_{s}}{K} ( 1 - {ⅇ}^{kt} )$$

$$K = {K}_{6} + {K}_{5} \frac{[ I ] / {K}_{I}}{1 + [ S ] / {K}_{m} + [ I ] / {K}_{I}}$$

$${V}_{s} = {V}_{o} \frac{1 + [ S ] / {K}_{m}}{1 + [ S ] / {K}_{m} + [ I ] / {K}_{i}^{ss}}$$

wherein

[P], [I], [S]=product, inhibitor, and substrate concentration

Vₛ=steady state rate

V_{z}=zero time rate

Vₒ=rate at [I]=0

Kₘ=Michaelis constant for the enzyme substrate pair (independently determined under the same experimental conditions)

Full details of the Experimental Protocol are reported in M. Alpegiani et al., Eur. J. Med. Chem. 1992, 27, 875-890.

Results

Table 2 reports the above defined “potency” and “efficiency” parameters for two representative compounds within the present invention, namely a compound of formula (I) wherein X is acyloxy (No. 1 in Table 1), and a compound of formula (I) wherein X is heterocyclyl-thio (No. 11 in Table 1), in comparison with a compound of the prior art, i.e. a compound of formula (I) wherein X is hydrogen (Reference). To make the comparison more meaningful, one of the beta-lactam derivatives emerging from the research on HLE-inhibitors by Merck Sharp & Dohme, code-named L-659,286, whose biochemistry and animal pharmacology has been extensively reported (Bonney et al. 1989, 39, 47-53), has been selected as the Reference.

Table 2

Kinetic parameters for HLE inhibition (see Protocol) by two representative compounds of the present invention, and a Reference compound of the prior art (Merck L-659,286) selected for structural similarity and recognized HLE-inhibitory activity

| | “Potency” | “Efficiency” |
|---|---|---|
| COMPOUND | Kᵢ^{ss} (nM) | k₅/Kᵢ (M⁻¹s⁻¹) |
| Compound No. 1 | <2 | 800,000 |
| Compound No. 11 | 20 | 27,000 |
| Reference | 100 | 9,200 |

The potentialities of protease inhibitor therapy in the treatment of conditions resulting from the destruction of connective tissues have recently received particular attention. Much effort has been devoted to the search for inhibitors of human leukocyte elastase (HLE), which is thought to be the primary destructive agent in pulmonary emphysema, may play important roles in rheumatoid arthritis, and is probably involved in the self-perpetuation of inflammatory cycles which characterizes several pulmonary ailments (J. C. Powers, Am. Rev. Resp. Dis. 127, S54-S58, 1983; C. H. Hassal et al, FEBS Letters, 183, n. 2, 201, 1985, G. Weinbaum and V.V. Damiano, TIPS, 8, 6, 1987; M. Velvart, Rheumatol. Int. 1, 121, 1981). Low molecular weight inhibitors appear to have a number of advantages over natural high molecular weight protease inhibitors from either plant or animal sources: 1) they can be obtained in quantities; 2) they can be rationally designed or optimised; 3) they are not antigenic; and 4) they may be used orally or in aerosols. Many low molecular weight elastase inhibitors discovered so far contain reactive functional groups (chloromethyl ketones, isocyanates, etc); they may react with functional groups of proteins, and therefore they may be quite toxic. In this respect, -lactam compounds are of potential interest because, though reactive towards some serine protease, they are, as it is known, non-toxic at very high concentrations.

Owing to their high elastase-inhibiting activity and their quite negligible toxicity, the compounds of the present invention can be used in mammals, including humans, for the prevention and treatment of inflammatory and degenerative diseases where elastases, in particular HLE, are, in any step of the disease etiology, progression or sustainment, involved. Thus, the compounds can be used to make medicaments useful to prevent or arrest the proteolytic degradation of lungs and connective tissues, reduce inflammation, reduce bronchial hypersecretions, and relieve pain. Diseases in which these compounds may find use are chronic diseases such as pulmonary emphysema, cystic fibrosis, bronchiectasis, bronchial inflammation, chronic obstructive pulmonary disease (COPD), rheumatoid arthritis, inflammatory bowel disease; and acute diseases, such as acute respiratory distress syndrome and septic and thraumatic shocks. Accordingly, the present invention also provides pharmaceutical and veterinary compositions containing a suitable carrier and/or diluent and, as an active principle, a 2-substituted 1,1-dioxo cephem amide, ester or thioester of formula (I) or a pharmaceutically or veterinarily acceptable salt thereof. The pharmaceutical or veterinary compositions containing a compound of formula I or salt thereof may be prepared in a conventional way by employing conventional non-toxic pharmaceutical carriers or diluents in a variety of dosage forms and ways of administration. In particular, the compounds of formula I can be administered:

A) Orally, for example, as tablets, troches, lozenges, aqueous or oily suspensions, dispersible powders or granules, emulsions, hard or soft capsules, or syrups or elixirs. Compositions intended for oral use may be prepared according to any method known in the art for the manufacture of pharmaceutical compositions and such compositions may contain one or more agents selected from the group consisting fo sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, maize starch, or alginic acid; binding agents, for example starch, gelatin or acacia, and lubricating agents, for example magnesium stearate, stearic acid or talc. The tablets may be uncoated or they may be coated by known techniques to delay disintegration and adsorption in the gastrointestinal tract and thereby provide a sustained action over a longer period. For example, a time delay material such as glyceryl monostearate or glyceryl distearate may be employed. Formulation for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin, or olive oil. Aqueous suspensions contain the active materials in admixture with excipients suitable for the manufacture of aqueous suspensions. Such excipients are suspending agents, for example, sodium carboxymethylcellulose, methylcellulose, hydroxy propylmethylcellulose, sodium alginate, polyvinylpyrrolidone gum tragacanth and gum acacia; dispersing or wetting agents may be naturally-occurring phosphatides, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyoxyethylene sorbitan monooleate. The said aqueous suspensions may also contain one or more preservatives, for example, ethyl or n-propyl p-hydroxybenzoate, one or more coloring agents, one or more flavoring agents, or one or more sweetening agents, such as sucrose or saccharin. Oily suspension may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents, such as those set forth above, and flavouring agents may be added to provide a palatable oral preparation. These compositions may be preserved by the addition of an antioxidant such as ascorbic acid. Dispersible powders and granules suitable for peparation of an aqueous suspension by the addition of water provide the active ingredient in admixture with a dispersing or wetting agent, a suspending agent and one or more preservatives. Suitable dispersing or wetting agents and suspending agents are exemplified by those already mentioned above. Additional excipients, for example sweetening, flavoring and coloring agents, may also be present. The pharmaceutical compositions of the invention may also be in the form of oil-in-water emulsions. The oily phase may be a vegetable oil, for example olive oil or arachis oils, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan mono-oleate, and condensation products of the said partial esters with ethylene oxide, for example polyoxyethylene sorbitan monooleate. The emulsion may also contain sweetening and flavoring agents. Syrups and elixirs may be formulated with sweetening agents, for example glycerol, sorbitol or sucrose. Such formulations may also contain a demulcent, a preservative and flavoring and coloring agents.

B) Parenterally, either subcutaneously, or intravenously, or intramuscularly, or intrasternally, or by infusion techniques, in the form of sterile injectable aqueous or olagenous suspensions. The pharmaceutical compositions may be in the form of a sterile injectable aqueous or olagenous suspension. This suspension may be formulated according to the known art using those suitable dispersing of wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition fatty acids such as oleic acid find use in the preparation of injectables;

C) By inhalation, in the form of aerosols or solutions for nebulizers;

D) Rectally, in the form of suppositories prepared by mixing the drug with a suitable non-irratating excipient which is solid at ordinary temperature but liquid at the rectal temperature and will therefore melt in the rectum to release the drug. Such materials are cocoa butter and poly-ethylene glycols;

E) Topically, in the form of creams ointments, jellies, solutions or suspensions. Still a further object of the present invention is to provide a method fo controlling inflammatory and degenerative diseases by administering a therapeutically effective amount of one or more of the active compounds encompassed by the formula I in humans or mammalians in need of such treatment. Daily dose are in the range of about 0.1 to about 50 mg per kg of body weight, according to the activity of the specific compound, the age, weight and conditions of the subject to be treated, the type and severity of the disease, and the frequency and route of administration; preferably, daily dosage levels for humans are in the range of 20 mg to 2 g. The amount of active ingredient that may be combined with the carrier materials to produce a single dosage form will vary depending upon the host treated and the particular mode of administration. For example, a formulation intended for the oral administration to humans, may contain from 5 mg to 2 g of active agent compounded with an appropriate and convenient amount of carrier material which may vary from about 5 to about 95 percent of the total composition. Dosage unit forms will generally contain between from about 5 mg to about 500 mg of active ingredient.

Experimental Part

### EXAMPLE 1

(6R,7S)-4-Bromo-7-methoxy-3-methyl-5,5-dioxo-2-(pyrrolidine-1-carbonyl)-5-thia-1-aza-byciclo[4.2.0]oct-2-en-8-one

A solution of (6R,7S)-7-methoxy-3-methyl-5,5-dioxo-2-(pyrrolidine-1-carbonyl)-5-thia-1-aza-bicyclo[4.2.0]oct-2-en-8-one (720 mg) in acetonitrile (30 ml) was treated with triethylamine (0.41 ml) an N-bromosuccinimide (530 mg). After stirring for 30 minutes, the reaction mixture was poured into EtOAc/water. The organic phase was collected and dried over Na₂SO₄. Removal of the solvent in vacuo left a crude residue which was purified by flash chromatography affording the title compound (720 mg) as a white foam. IR (CHCl₃) υ max 1800, 1745, 1720, 1640 cm⁻¹ NMR (200 MHz, CDCl₃) δ 1.89 (3H, s), 1.9-2.0 (4H, m), 3.3-3.6 (4H, m), 3.56 (3H, s), 4.90 (1H, s), 5.17 (1H, d, J=1.9 Hz), 5.30 (1H, d, J=1.9 Hz).

### EXAMPLE 2

(6R,7S)-7-Methoxy-3-methyl-4-(1-methyl-1H-tetrazol-5-ylsulfanyl)-5,5-dioxo-2-(pyrrolidine-1-carbonyl)-5-thia-1-az a-bicyclo[4.2.0]-oct-2-en-8-one (Compound 4)

To a solution of (6R,7S)-4-bromo-7-methoxy-3-methyl-5,5-dioxo-2-(pyrrolidine-1-carbonyl)-5-thia-1-aza-byciclo[4.2.0]oct-2-en-8-one (195 mg) in acetonitrile (10 ml) was added sodium 1-methyl-1,2,3,4-tetrazolyl-5-mercaptide dihydrate (170 mg). In about 30 minutes the reaction was over. The reaction mixture was diluted with E tOAc, sequentially washed with saturated NaHCO₃ and brine an dried over Na₂SO₄. Removal of the solvent iin vacuo left the crude title product which was obtained pure as a white solid by silica gel chromatography (150 mg).

IR (KBr) υ max 1790, 1640 cm⁻¹

NMR (200 MHz, CDCl₃) δ 1.9-2.0 (4H, m), 2.05 (3H, s), 3.2-3.7 (4H, m), 3.55 (3H, s), 4.09 (3H, s), 5.07 (1H, d, J=1.9 Hz), 5.18 (1H, d, J=1.9 Hz), 5.22 (1H, s).

### EXAMPLE 3

(6R,7S)-4-(6-Hydroxy-2-methyl-5-oxo-2,5-dihydro-[1,2,4]triazin-3-yl-sulfanyl)-7-methoxy-3-methyl-5,5-dioxo-2-(pyrrolidine-1-carbonyl)-5-thia-1-aza-bicyclo [4.2.0]oct-2-en-8-one (Compound 11).

A solution of (6R,7S)-4-bromo-7-methoxy-3-methyl-5,5-dioxo-2-(pyrrolidine-1-carbonyl)-5-thia-1-aza-bcyclo[4.2.0]oct-2-en-8-one (130 mg), prepared as in Example 1, in dry acetonytrile (10 ml) was treated with triethylamine (0.8 ml) and 3-mercapto-2-methyl-5-oxo-6-benzhydryloxy-2,5-dihydro-1,2,4 triazine (180 mg). After 4 hours the reaction mixture was partitioned between EtOAc/water. The upper phase was collected, dried over Na₂SO₄ and concentrated to give a residue which was purified by flash-chromatography, thereby obtaining the protected compound as a light yellow oil (130 mg).

It was dissolved in dichloromethane (3 ml) then anisole (0.02 ml) and trifluoroacetic acid (1 ml) were added. After 30 minutes TFA was completely removed in vacuo and the residue taken up in dichloromethane (1 ml). Addition of isopropylether afforded the title compound (60 mg) as a whitish powder.

IR (KBr) υ max 1795, 1650 (broad) cm⁻¹

NMR (200 MHz, DMSO-d₆) δ 1.82 (3H, s), 1.8-2.0 (4H, m), 3.2-3.7 (4H, m), 3.45 (3H, s), 3.69 (3H, s), 5.41 (1H, d, J=1.6 Hz), 5.83 (1H, d, J=1.6 Hz), 6.01 (1H, s).

### EXAMPLE 4

2,4,6-Trimethylbenzoic Acid (6R,7S)-7-methoxy-3-methyl-5,5,8-trioxo-2-(pyrrolidine-1-carbonyl)-5-thia-1-aza-bicyclo[4.2.0] oct-2-en-4-yl Ester (Compound 1).

To a solution of (6R,7S)-4-bromo-7-methoxy-3-methyl-5,5-dioxo-2-(pyrrolidine-1-carbonyl)-5-thia-1-aza-bicyclo[4.2.0]oct-2-en-8-one (195 mg) (prepared according the procedure described in example 1) in acetonitrile (10 ml), 2,4,6-trimethyl silver benzoate (205 mg) was added and the resulting mixture was stirred at room temperature for 30 minutes. Silver bromide was filtered off and the solution diluted with EtOAc and eventually washed with water. Upon drying over Na₂SO₄, the organic solvent was rotoevaporated and flash chromatography of the crude residue gave the pure title product as a light yellow powder (130 mg).

IR (KBr) υ max 1800, 1755, 1650 cm⁻¹

NMR (200 MHz, CDCl₃) δ 1.88 (3H, s), 1.8-2.0 (4H, m), 2.31 (3H, s), 2.35 (6H, s), 3.2-3.7 (4H, m), 3.56 (3H, s), 4.75 (1H, d, J=1.9 Hz), 5.20 (1H, d, J=1.9 Hz), 6.09 (1H, s), 6.90 (2H, s).

### EXAMPLE 5

(6R,7S)-4-Bromo-7-methoxy-3-methyl-5,5,8-trioxo-5-thia-1-aza-bicyclo[4.2.0]oct-2-ene-2-carboxylic Acid Tert-Butyl Ester.

Starting from a solution of (6R,7S)-7-methoxy-3-methyl-5,5,8-trioxo-5-thia-1-aza-bicyclo[4.2.0]oct-2-ene-2-carboxylic acid tert-butyl ester (350 mg), triethylamine (0.15 ml) and N-bromosuccinimide (180 mg) in acetonitrile (20 ml) and following the procedure described in Example 1, the title compound was obtained pure as a light yellow foam (280 mg).

IR (KBr) υ max 1810, 1720 cm⁻¹

NMR (200 MHz, CDCl₃) δ 1.55 (9H, s), 2.08 (3H, s), 3.58 (3H, s), 4.92 (1H, s), 5.14 (1H, d, J=1.8 Hz), 5.25 (1H,d, J=1.8 Hz).

### EXAMPLE 6

(6R,7S)-4-Benzoyloxy-7-methoxy-3-methyl-5,5,8-trioxo-5-thia-1-aza-bicyclo[4.2.0]oct-2-ene-2-carboxylic Acid Tert-Butyl Ester (Compound 7).

To a solution of (6R,7S)-4-bromo-7-methoxy-methyl-5,5,8trioxo-5-thia-1-aza-bicyclo[4.2.0]oct-2-ene-2carboxylic acid tert-butyl ester (270 mg) in acetonitrile (10 ml) silver benzoate (250 mg) was added and the mixture was stirred for 1 hour. Silver bromide was removed by filtration and the solution was diluted with EtOAc, washed with water and eventually with brine. Upon drying over Na₂SO₄, the organic solvent was rotoevaporated and the crude residue was flash chromatographed over SiO₂. The title compound was obtained pure as a white solid (190 mg).

IR (KBr) υ max 1805, 1745, 1730 cm⁻¹

NMR (200 MHz, CDCl₃) δ 1.58 (9H, s), 2.00 (3H, s), 3.58 (3H, s), 4.78 (1H, d, J=1.6 Hz), 5.19 (1H, d, J=1.6 Hz), 6.03 (1H, s), 7.3-8.1 (5H, m)

### EXAMPLE 7

(6R,7S)-7-Methoxy-3-methyl-4-(1-methyl-1H-tetrazol-5-ylsulfanyl)-5,5,8-trioxo-5-thia-1-aza-bicyclo[4.2.0]oct-2-ene-2-carbothioic Acid S-tert-butyl Ester (Compound 6).

A solution of toluene-4-thiosulfonic acid S-(1-methyl-1H-tetrazol-5-yl) ester (50 mg) and (6R,7S)-7-methoxy-3-methyl-5,5,8-trioxo-5-thia-1-aza-bicyclo[ 4.2.0]oct-2-ene-2-carbothioic acid S-tert-butyl ester (50 mg) in acetonitrile (2.5 ml) was treated with 1,5-diazabicyclo[4,3,0]non-5-ene (25 μl) and let stand at room temperature for 2 hours. The reaction mixture was poured into EtOAc/0.5N hydrochloric acid. The organic phase was washed twice with water, dried (Na₂SO₄) and concentrated under vacuum. The residue was flash chromatographed over SiO₂ eluting with EtOAc/n-hexane 1:1. The title product was obtained as a white foam (48 mg).

IR (KBr) υ max 1800, 1660 cm⁻¹

NMR (200 MHz, CDCl₃) δ 1.63 (9H, s), 2.22 (3H, s), 3.65 (3H, s), 4.16 (3H, s), 5.14 (1H, d, J=1.9 Hz), 5.19 (1H, s), 5.24 (1H, d, J=1.9 Hz).

### EXAMPLE 8

Benzoic Acid (6R,7S)-2-tert-butylsulfanylcarbonyl-7-methoxy-3-methyl-5,5,8-trioxo-5-thia-1-aza-bicyclo [4.2.0]oct-2-en-4-yl Ester (Compound 8).

1,5-Diazabicyclo[4,3,0]non-5-ene (50 μl) in acetonitrile (0.5 ml) was added dropwise to a solution of benzoyl peroxide (80 mg) and (6R,7S)-7-methoxy-3-methyl-5,5,8-trioxo-5-thia-1aza-bicyclo[4.2.0]oct-2-ene-2-carbothioic acid S-tert-butyl ester (100 mg) in acetonitrile (2.5 ml), while keeping the temperature of the reaction mixture under 30° C. After stirring for 30 minutes at room temperature, the reaction mixture was partitioned between EtOAc and 1% hydrochloric acid. The upper phase was sequentially washed with water, aqueous NaHCO₃ and brine. Following chromatographic purification, the title product was obtained as a waxy solid.

IR (CHCl₃) υ max 1810, 1745, 1660 cm⁻¹

NMR (200 MHz, CDCl₃) δ 1.57 (9H, s), 1.93 (3H, s), 3.58 (3H, s), 4.78 (1H, d, J=1.8 Hz), 5.19 (1H, d, J=1.8 Hz), 6.02 (1H, s), 7.4-8.2 (5H, m).

### EXAMPLE 9

1-[(6R,7S)-7-Methoxy-3-methyl-4-(1-methyl-1H-tetrazol-5-yl-sulfanyl)-5,5,8-trioxo-5-thia-1-aza-bicyclo[4.2.0]oct-2-enane-2-carbonyl]-pyrrolidine-2(S)-carboxylic Acid (Compound No 3).

A solution of (6R,7S)-7-methoxy-3-methyl-5,5,8-trioxo-5-thia-1-aza-bicyclo[4.2.0]oct-2-enane-2-carbonyl]-pyrrolidine-2(S)-carboxylic acid benzhydryl ester (1.5 g) in acetonitrile (30 ml) was treated, at room temperature, with triethylamine (0.51 ml) and N-bromosuccinimide (0.66 g). After 1 h, the solvent was removed in vacuo. The residue was washed with a saturated aqueous solution of Na₂S₂O₅ and then purified by flash chromatography, thereby obtaining 1-[(6R,7S)-4-bromo-7-methoxy-3-methyl-5,5,8-trioxo-5-thia-1-aza-bicyclo[4.2.0]-oct-2-enane-2-carbonyl]-pyrrolidine-2(S)-carboxylic acid benzhydryl ester as a white powder (650 mg). A portion of this product (mg 200) was dissolved in dimethylformamide (5 ml) and treated with sodium 1-methyl-1,2,3,4-tetrazolyl-5-mercaptide dihydrate (115 mg). After 10 minutes, the reaction mixture was diluted with AcOEt and washed with brine. Removal of the solvent afforded a yellowish solid which was treated with a mixture of methylene chloride (2.4 ml), trifluoroacetic acid (2.2 ml) and anisole (1.2 ml). After 15 minutes TFA was removed in vacuo and the oily residue was taken up with diisopropylether. Collection of the precipitate by filtration afforded the pure title compound (72 mg) as a light brown solid. MS (FD): 472 m/z (molecular peak).

### EXAMPLE 10

1-[(6R,7S)-7-Methoxy-3-methyl-5,5,8-trioxo-4-(2,4,6-trimethyl-benzoyloxy)-5-thia-1-aza-bicyclo[4.2.0]oct-2-enane-2-carbonyl]-pyrrolidine-2(S)-carboxylic Acid (Compound 2).

A solution of 1-[(6R,7S)-4-bromo-7-methoxy-3-methyl-5,5,8-trioxo-5-thia-1-aza-bicyclo[4.2.0]oct-2-enane-2-carbonyl]-pyrrolidine-2(S)-carboxylic acid benzhydryl ester (280 mg) in acetonitrile (23 ml) was treated with silver 2,4,6-trimethylbenzoate (220 mg). After 10 minutes, the reaction was complete: silver bromide was filtered off and the solution concentrated to residue in vacuo. The residue was diluted with AcOEt and washed with NaHCO₃ aq. and water. The organic layer was concentrated to dryness: the residue was purified by flash chromatography, thereby obtaining the bezhydryl ester of the title compound (300 mg), which was treated with a mixture of dichloromethane (3.2 ml), trifluoroacetic acid (3.2 ml) and anisole (1.6 ml). After 15 minutes, the solution was concentrated in vacuo. The residual oil was dissolved in diethylether and the addition of cyclohexane afforded the precipitation of the title compound (88 mg) as a mixture (4:1) of the two amide rotamers.

NMR major product (400 MHz, CDCl₃) d 1.93 (3H, s)₁ 1.8-2.4 (4H, m), 2.31 (3H, s), 2.36 (6H, s), 3.45 (2H, m), 3.55 (3H, s), 4.69 (1H, dd, j=3.5, 8.5 Hz), 4.78 (1H, d, j=1.8 Hz), 5.23 (1H, d, j=1.8 Hz), 6.04 (s, 1H), 6.91 (2H, s).

1. A compound of the formula I and the pharmaceutically and veterinarily acceptable salts thereof: 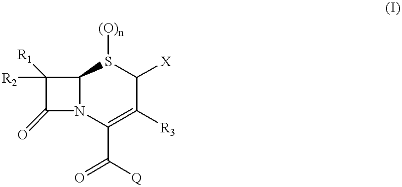
wherein n is one or two;
X is
(1) a heterocyclyl-thio group, wherein the heterocyclyl group is an optionally substituted 3-6 membered, saturated or unsaturated heterocyclic ring, containing at least one heteroatom selected from O, S and N, which is optionally fused to a second 5-6 membered, saturated or unsaturated heterocyclyl group, or to a C₃-C₈ cycloalkyl group, or to a cyclopentenyl group, or to a C₆-C₁₀ aryl group;
(2) an acyloxy group —O—C(O)A wherein A is an organic radical selected from C₁-C₁₂ straight or branched alkyl, C₂-C₁₂ alkenyl, C₂-C₁₂ alkynyl, C₆-C₁₄ aryl, C₃-C₈ cycloalkyl, C₅-C₈ cycloalkenyl, or C₇-C₁₈ aralkyl, C₁-C₁₈ aralkenyl, C₁-C₁₈ aralkynyl, (cycloalkyl) alkyl, (cycloalkyl)alkenyl, heterocyclyl, (heterocyclyl)alkyl, (heterocyclyl)alkenyl;
Q represents a group —OA, —SA or —NAA′ wherein A is as defined above and A′ is hydrogen or, being the same or different, is as defined above for A; or A and A′ taken together with the nitrogen atom to which they are attached represent a 5-7 membered ring optionally containing an additional heteroatom selected from N, O and S; R¹ and R² independently represent
(1) hydrogen, chloro, fluoro, bromo or iodo;
(2) A as defined above;
(3) hydroxy —OH or an ether —OA wherein A is as defined above;
(4) a thioether, sulfoxide or sulfone —S(O)ₘA wherein m is either zero, one or two and A is as defined above;
(5) acyloxy —OC(O)A wherein A is as defined above;
(6) acyl —C(O)A or —C(O)OA wherein A is as defined above;
(7) sulfonyloxy —OS(O)₂A wherein A is as defined above;
(8) acetylamino or trifluoroacetamido, or an acylamino group ZNH—CHR—CONH—, wherein R is methyl, ethyl, isopropyl Me₂CH—, EtMeCH—, Me₂CHCH₂—, EtMeCHCH₂, and Z is either hydrogen or:
phenylcarbonyl or phenoxymethylcarbonyl or phenoxy-methylcarbonyl, wherein the phenyl ring is either unsubstituted or substituted by one or two chloro, fluoro or methyl groups, or by a group selected among the following ones:
a) carboxy
b) OCH₂CO₂H
c) OCH₂CH₂-4-morpholinyl
d) OCH₂CH₂-1-pyrrolidinyl
e) SO₂-1-morpholinyl
f) SO₂-(4-methyl)-1-piperazinyl
g) SO₂N(Me)CH₂CH₂NMe₂
h) CONH—CO-1-morpholinyl
i) CO-1-morpholinyl
j) CO-(4-methyl)-1-piperazinyl
k) CONH—CH₂CH₂-1-morpholinyl
l) COO—CH₂CH₂-1-morpholinyl
m) SO₂NH—Ar, or CONHSO₂—Ar wherein Ar is a phenyl ring either unsubstituted or substituted by Cl, F or carboxy, said carboxy being optionally as the ethyl ester COOC₂H₅ or amides CONH₂, CONHCH₃, CONHCH₂ CO₂H,
an amino-blocking group, selected from the group consisting of e), f), g), i), k), l) above or from the following ones: methyl, dimethyl, benzyl, CO₂CH₃, COSCH₃; SO₂CH₃;
(9) azido, nitro or cyano;
or R¹ and R² taken together constitute an oxo group (═O) or a group of formula ═CHA′, ═CHC(O)A′, ═CHC(O)OA′ or ═CHS(O)₂A wherein A and A′ are as defined above;
R³ represents:
(1) A′ as defined above;
(2) chloro or fluoro;
(3) a sulfenyl, sulfinyl or sulfonyl group —S(O)ₘA wherein m and A are as defined above;
(4) an ether group —O—A wherein A is as defined above;
(5) an acyl group —C(O)A, —C(O)OA or —CO₂H wherein A is as defined above;
(6) an oxymethyl group —CH₂—OA′ wherein A′ is as defined above;
(7) a thiomethyl group or a derivative thereof of formula CH₂S(O)ₘA wherein m and A are as defined above;
(8) an acyloxymethyl group —CH₂OC(O)A′ wherein A′ is as defined above;
(9) an acylthiomethyl group —CH₂SC(O)A wherein A is as defined above;
(10) carbamoyloxymethyl —CH₂OCONH₂ or carbamoylthiomethyl (i.e., —CH₂SCONH₂), and the N-methyl and N,N-dimethyl derivatives thereof;
(11) an aminomethyl group —CH₂—N(A)A′ wherein A and A′ are as defined above;
(12) ammoniomethyl —CH₂N⁺(A)(A′)A″ wherein A and A′ are as defined above and A″, being the same or different, is as defined for A; or A is alkyl and A′ and A″ together with the nitrogen atom to which they are attached represent a 5-7 membered ring containing one nitrogen and optionally one oxygen or sulfur atom;
(13) an acylaminomethyl group —CH₂NH—C(O)A wherein A is as defined above.

2. A compound or salt according to claim 1 wherein:
n is two, X is
(1′) an optionally substituted heterocyclyl-thio group, wherein the heterocyclyl group is an unsaturated heterocyclyl ring chosen among pyrrolyl, pyrazolyl, imidazolyl, triazolyl, tetrazolyl, oxazolyl, isoxazolyl, thiazolyl, isothiazolyl, thiadiazolyl, thienyl, furyl, pyridinyl, pyrazinyl, pyrimidinyl, triazinyl, pyridazinyl, benzothienyl, benzothiazolyl, benzoxazolyl, isobenzofuranyl, benzofuranyl, chromenyl, indolyl, indolizinyl, isoindolyl, cinnolinyl, indazolyl, purinyl, benzopyridyl, benzopiperidyl, (cyclopentano)pyridyl, (cyclopenteno)pyridyl, (tetrazolo)pyridazinyl;
(2′) a group —OC(O)A wherein A is chosen among an optionally substituted C₁-C₁₂ straight or branched alkyl, C₂-C₁₂ straight or branched alkenyl, C₂-C₁₂ alkynyl, C₃-C₆ cycloalkyl, C₆-C₁₄ aryl, benzyl, diphenylmethyl, stiryl, 2-phenyl-2-propyl; or an optionally substituted heterocyclyl, wherein the heterocyclyl group is either one of the unsaturated heterocyclyl groups specified under (1′) immediately above, or it is a 3-6 membered saturated ring containing 1-3 heteroatoms selected from N, O and S, preferably pyrrolinyl, aziridinyl, piperidyl, oxiranyl, tetrahydro-pyranyl, morpholinyl; the substituents for the heterocyclyl groups and for the groups A defined above being selected from fluoro, chloro, bromo, nitro, cyano, sulfo, carboxy, tetrazolyl, C₁-C₄ alkoxycarbonyl, carbamoyl, sulfamoyl, carbamoyloxy, hydroxy, C₁-C₄ alkoxy, benzyloxy, benzhydryloxy, phenoxy, acetoxy, pivaloyloxy, benzoxy, methylthio, phenylthio, methansulfonyl, benzensulfonyl, sulfomethyl, carboxymethyl, carboxyethyl, carboxypropyl, carboxymethylthio, carboxyphenyl C₆H₅—COOH, carboxybenzyl CH₂—C₆H₅—COOH, acetyl, trifluoroacetyl, benzoyl, pivaloyl, amino, dimethylamino, diethylamino, dimethylaminoethyl, formamido, acetamido, trifluoroacetamido, pivalamido, oxo, C₁-C₅ straight or branched alkyl, vinyl and allyl;
Q represents a group —OA, —SA or —NAA′ wherein A is defined above and A′ is hydrogen or, being the same or different, is as defined above for A; or A and A′ taken together with the nitrogen atom to which they are attached represent a saturated 5-6 membered ring optionally containing an additional heteroatom selected from O, S, and N; said ring or said groups A and A′ being unsubstituted or substituted by one or more of the substituents detailed above;
R¹ and R², each independently, are
(1′) hydrogen, chloro, fluoro or bromo;
(2′) straight or branched C₁-C₅ alkyl or C₁-C₅ alkenyl;
(3′) C₁-C₅ alkyloxy or C₆-C₁₀ aryloxy;
(4′) C₁-C₅ alkylthio or C₁-C₁₀ arylthio;
(5′) C₁-C₅ alkylsulfinyl, C₂-C₅ alkenylsulfinyl,
(6′) C₁-C₅ alkylsulfonyl or C₆-C₁₀ arylsulfonyl;
(7′) C₁-C₅ alkylcarbonyl or C₆-C₁₀ arylcarbonyl;
(8′) C₁-C₅ alkylcarbonyloxy or C₆-C₁₀ arylcarbonyloxy;
(9′) C₁-C₅ alkylsulfonyloxy or C₆-C₁₀ arylsulfonyloxy;
(10′) acetamido or trifluoroacetamido;
(11′) nitro, azido, cyano, formyloxy;
(12′) an acylamino group ZNH—CHR—CONH derived from an L-aminoacid, wherein Z is
phenoxycarbonyl
4-chlorophenoxycarbonyl
benzyloxycarbonyl
4-chlorobenzyloxycarbonyl
4-[(1-morpholinyl)carbonyl]benzyloxycarbonyl
(1-morpholinyl)carbonyl
4-[(2-carboxyphenylaminosulfonyl]benzylocarbonyl;
4-[(4-chloro)phenylsulphonylamino]benzoyl;
and R is as defined in claim 1;
or R¹ and R² taken together constitute an oxo group, or a methylene group, or a group of formula ═CHY or ═CHC(O)Y or ═CHC(O)OY or ═CHS(O)₂Y, wherein Y is C₁-C₅ alkyl, C₁-C₅ alkenyl, C₆-C₁₀ aryl, C₇-C₁₀ aralky or heterocyclyl, wherein heterocyclyl is one of the heterocyclyl groups detailed above in the definition (1′) of X; the substituents for the groups defined under (2′)-(9′) being selected from fluoro, chloro, bromo, nitro, cyano, sulfo, carboxy, tetrazolyl, C₁-C₄ alkoxycarbonyl, carbamoyl, sulfamoyl, carbamoyloxy, methansulfonyl, hydroxy, C₁-C₄ alkoxy, benzyloxy, benzhydryloxy, phenoxy, acetoxy, pivaloyloxy, benzoxy, methylthio, phenylthio, methansulfonyl, benzensulfonyl, sulfomethyl, carboxymethyl, carboxyethyl, carboxypropyl, carboxymethylthio, carboxyphenyl C₆H₅—COOH, carboxybenzyl CH₂—C₆H₅—COOH, acetyl, trifluoroacetyl, benzoyl, pivaloyl, amino, dimethylamino, diethylamino, dimethylaminoethyl, formamido, acetamido, trifluoroacetamido, pivalamido, oxo, C₁-C₅ straight or branched alkyl, vinyl and allyl;
R³ is either hydrogen or
(1′) methyl, ethyl, propyl, phenyl or benzyl, optionally substituted by a group selected from chloro, bromo, fluoro, hydroxy, carbamoyloxy, carboxy;
(2′) chloro;
(3′) methylthio;
(4′) methoxy or benzyloxy;
(5′) formyl, acetyl, benzoyl, carboxy, methoxycarbonyl, ethoxycarbonyl, tert-butoxycarbonyl or benzyloxycarbonyl;
(6′) methoxymethyl, ethoxymethyl, isopropoxymethyl; or benzyloxymethyl, phenoxymethyl, 3-pyridyloxymethyl wherein the phenyl and pyridyl rings are either unsubstituted or substituted by one group or two equal or different groups chosen from hydroxy, carboxy, amino, halogen and C₁-C₄ alkoxycarbonyl;
(7′) methylthiomethyl, methylsulfinylmethyl or methylsulfonyl-methyl; or a heterocyclylthiomethyl group wherein the heterocyclyl ring is one of those listed above in the definition (1′) of X, said groups being either unsubstituted or substituted by one or two, equal or different groups chosen from the following ones: hydroxy, oxo, amino, imino, methylamino, dimethylamino, acetylamino, sulfo, carboxy, carbamoyl, methylcarbamoyl, dimethylcarbamoyl, carbamoyloxy, dimethylaminomethyl, carboxymethyl, carboxymethylthio, cyano, cyanomethyl, nitro, methoxy, phenoxy, benzyloxy, benzhydryloxy, methylthio, methylsulfonyl, acetoxy, benzoxy, halogen or C₁-C₄ alkyl or alkenyl;
(8′) acetoxymethyl, benzoyloxymethyl, phenylacetoxymethyl or C₃-C₆ alkanoyloxymethyl wherein the above groups are either unsubstituted or substituted by one or more groups selected from carboxy, carboxymethyl, hydroxy, C₁-C₃ alkoxy, carbamoyl;
(9′) carbamoyloxymethyl —CH₂OCONH₂;
(10′) trialkylammoniomethyl wherein the alkyl group is chosen from methyl, ethyl or propyl; N-methylpyrrolidiniomethyl; N-methylpiperidiniomethyl; N-methylmorpholiniomethyl; pyridiniomethyl which is either unsubstituted or substituted on the heterocyclic ring by fluoro, chloro, methoxy, hydroxy, carboxy or carbamoyl;
and the pharmaceutically and veterinarily acceptable salts thereof and all of the possible stereoisomers e.g.: epimers, diastereoisomers, geometrical isomers, tautomers.

3. A process for preparing a compound of-the formula I or salts thereof as defined in claim 1 or 2 which process comprises
(i) reacting a compound of formula (II) 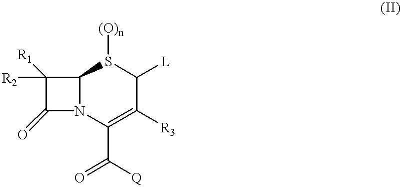
wherein either
(iₐ) n, Q, R¹, R² and R³ are as defined in claim 1, and L is a leaving group, with compounds of formula (III)
X-M (III)
wherein X is as defined in claim 1 and M is hydrogen or a metal; or
(i_{b}) n, Q, R¹, R² and R³ are as defined above, and L is hydrogen, with compounds of formula (IV)
X-X′ (IV)
wherein X is as defined above and X′, being the same or different is as defined above for X, or a group selected from halogen, i.e. fluorine, chlorine, bromine or iodine, a C₁-C₈ alkylsulfonyl, an arylsulfonyl, an imido group, or a leaving group of formula —OC(O)A, —OC(O)OA, —OS(O)₂A, —OC(O)NR^{iv}A wherein A is as defined in claim 1 and R^{iv} is phenyl or a C₁-C₄ alkyl group;
(ii) if desired, oxidising a resulting compound of formula (I) wherein n is one into the corresponding compound wherein n is two;
(iii) if desired, converting the resulting compound of formula (I) into a pharmaceutical or veterinarily acceptable salt thereof.

4. A process according to claim 3 characterized in that for carrying out the step referred to under (ia), the leaving group is a halogen atom and the reaction is performed in the presence of an inorganic or organic base, when M is hydrogen atom, in an organic solvent at a temperature of from −50° to 120° C.

5. A process according to claim 3 characterized in that the step referred to under (ib) is carried out in the presence of tertiary alyphatic or aromatic bases in non-protic organic solvents at a temperature of from −60° to 40° C.

6. A process according to claim 3 characterized in that the oxidation step referred to under (ii) is carried out with inorganic or organic peracids or salts thereof in an inert organic solvent or in a mixture of water and an organic solvent at a temperature of from −40° C. to +40° C.

7. A pharmaceutical or veterinary composition comprising, as an active ingredient, a compound of the formula I as defined in claim 1 or a pharmaceutically or veterinarily acceptable salt thereof and a pharmaceutically or veterinarily acceptable diluent or carrier.
